(19) United States
(12) Patent Application Publication
(10) Pub. No.: US 2016/0106428 A1
(43) Pub. Date: Apr. 21, 2016

(54) Anastomat and Closure Device with a Pressure Sensor and Automatic Adjustment

(71) Applicants: Yifu JIN, Nanjing (CN); Heiying JIN, Nanjing (CN)

(72) Inventors: Yifu JIN, Nanjing (CN); Heiying JIN, Nanjing (CN); Li CHEN, Nanjing (CN)

(73) Assignees: Yifu Jin, Nanjing (CN); Heiying JIN, Nanjing (CN)

(21) Appl. No.: 14/669,640

(22) Filed: Mar. 26, 2015

(30) Foreign Application Priority Data
Oct. 21, 2014 (CN) 201410562547.0

(51) Int. Cl. A61B 17/072; A61B 19/00

(52) U.S. Cl. CPC A61B 17/072; A61B 19/30; A61B 2017/00017

(57) ABSTRACT

An anastomat and closure device having a pressure sensor and automatic adjustment means, that a pressure value from direct or indirect contact of a stapling apparatus and a staple holder can be transmitted and displayed by a pressure sensor. The pressure sensor is installed on either side or on both sides of the stapling apparatus and the staple holder and wherein the pressure is displayed through a pressure sensor shaping circuit, an amplifying circuit, and a display circuit. The benefit of this invention is that surgical operators can intuitively and directly understand the pressure of the anastomotic tissue and determine whether the pressure is appropriate.

### TECHNICAL FIELD

This invention involves the structure of a surgical anastomat.

### BACKGROUND AND PRIOR ART

When conducting gastrointestinal anastomosis and closure, anastomotic bleeding and leakage are serious surgical complications. One of the main causes of anastomotic bleeding and leakage is distal and proximal pressure of the anastomosis (particularly when it involves two tissue types) that is too large, too small, or uneven. The lack of pressure (i.e., the two tissue layers are not joined) may lead to incomplete anastomosis or anastomotic bleeding. By contrast, excessive pressure (i.e., the pressure between the two layers is too large) will cause anastomotic necosis, thus resulting in anastomotic leakage and bleeding. Uneven pressure may cause either partially incomplete anastomosis or necrosis, which may result in anastomotic bleeding and leakage. The long-term consequences of the complications may include anastomotic stenosis and anastomotic failure. Therefore, the key to consistent success of gastrointestinal anastomosis is to maintain uniform pressure at anastomosis. However, all existing anastomats control anastomosis pressure by regulating the thickness of the proximal and distal anastomosis (the current technology works by measuring the tissue wall thickness). It is well known that there are large variations in the thicknesses of the gastrointestinal tract among individuals. Thus, it is sometimes difficult to control the pressure simply by thickness. In addition, the accuracy of the measurement cannot be guaranteed. Therefore, gastrointestinal anastomotic bleeding leakage complications are still relatively common.

Typical anastomats, such as utility model patent publication CN201410247371, are cutter anastomats. They include positioning handles, firing buttons, heel blocks, thrust plates, connection parts and cutting blades. The firing buttons are on either side of the heel blocks. The left side has the left firing button, and the right side has the right firing button. The two firing buttons are connected by beams, which perforate the connection between the heel blocks. The structure of the anastomat is simple, and it is easy to operate by either hand or by both hands. A pin connects the firing button and beam. Additionally, the firing button rotates inside or outside axially around the pin. The implementation of a rotating firing button reduces the overall size of the instrument, thereby minimizing packaging size, reducing cost, and facilitating transportation.

Utility model patent publication CN201310333489 is a cylindrical anastomat that consists of an anastomat body, an anvil assembly, a connecting staple barrel, and a projecting anvil rod from the end surface of the staple barrel. The anvil rod selectively connects to the anvil assembly. The described anvil rod includes at least part of the flexible portion of the described staple barrel and the connecting portion of the distal end of the described flexible portion. The connection between the described connecting portion and described anvil assembly can be removed.

To ensure the quality of the anastomosis and to reduce anastomotic bleeding and leakage complications after gastrointestinal anastomosis, it is necessary to design an anastomat with a pressure sensor and automatic adjustment means. However, such a device has not been published.

### SUMMARY OF THE INVENTION

The purpose of this invention is to design an anastomat with a pressure sensor and automatic regulation devices, thus reducing complications that may occur after gastrointestinal anastomosis such as anastomotic bleeding and leakage.

The technical plan of this invention is to make an anastomat with a pressure sensor and automatic adjustment means. Pressure from direct or indirect contact between the stapling apparatus and staple holder is applied and displayed by the pressure sensor.

The pressure sensor may be installed on either side or on both sides of the stapling apparatus and staple barrel. The signals are shown on the display circuit after pressure sensor shaping and amplification. Amplification is performed using an operational amplifier and is displayed after analog-to-digital (AD) conversion on a digital display circuit. We do not exclude communicating and displaying the pressure in the operating room screen wirelessly, or displaying the pressure value on the monitor of the handle by transmitting the signals through the central rod.

Similar to a cylindrical anastomat, our anastomat can transmit the pressure data from the center rod to the monitor at the handle. This allows the operator to directly understand the pressure of the anastomosis and determine whether the pressure is appropriate. Furthermore, the stapling apparatus and staple holder of the anastomat close gradually from the center to the periphery and adjust themselves according to the proximal and distal pressure measured by the pressure sensor. The distance between the stapling apparatus and staple holder will then be automatically adjusted to the set pressure level, thus achieving optimal thickness between the adjustor itself and the anastomat, as well as uniform pressure at the anastomosis.

The benefits of this invention include the following: surgical operators can intuitively and directly understand the pressure of the anastomotic tissue and determine whether the pressure is appropriate. In addition, when the anastomosis procedure involves cutting, the stapling apparatus of the cutting anastomat (stapler) and staple holder close gradually from the center to the periphery, control the proximal and distal pressure by the pressure sensor, and automatically adjust the height of the needle holder according to the set pressure. This process adjusts the thickness of the anastomosis automatically and helps achieve uniform pressure in anastomosis. The invention ensures consistent quality in anastomosis and reduces gastrointestinal anastomosis complications, including anastomotic bleeding and leakage. This is a type of anastomat that senses pressure and adjusts automatically.

### BRIEF DESCRIPTION OF THE DRAWINGS

FIG. 1 is a scheme of the structure of this invention.

### DETAIL DESCRIPTION

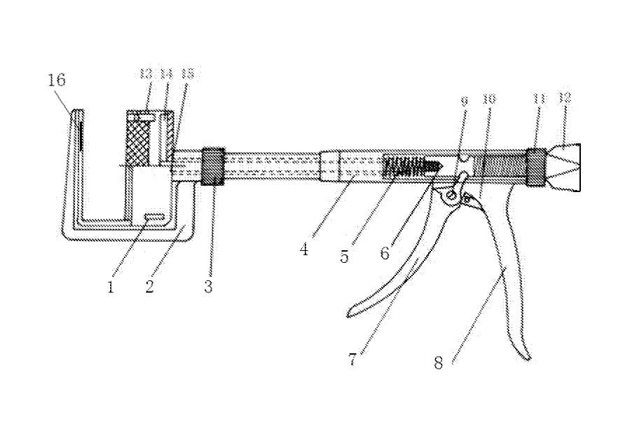

FIG. 1 is a linear anastomat. Structures of tubular anastomat can also be completely referred to FIG. 1.

Reference numbers in the FIGURE: 1—positioning knob; 2—bow; 3—tightening nut; 4—overtube; 5—return spring; 6—heel blocks; 7—left handle; 8—right handle; 9—shaft screw; 10—safety button; 11—empennage screw knob; 12—adjustment screw; 13—plastic components; 14—staple pusher; 15—assembly frame; 16—pressure sensor chip.

The prior art has disclosed a gastrointestinal anastomosis-mechanical suture method (anastomat method). The gastrointestinal anastomat procedure for gastrointestinal surgery can also be called stapler-style gastrointestinal anastomosis (sewing) technology. Intestinal anastomats include the LDS (Ligating-Dividing Stapling) anastomat for ligating the mesometrium and greater omentum vessels, the TA (Thoracic-Abdominal) anastomat for gastrointestinal tract suture, the GIA (Gastro-Intestinal Anastomosis) anastomat for gastrointestinal side-to-side anastomosis, and the EEA (End-End Anastomosis) cylinder anastomat. The USSC (United States Surgical Corporation) has launched a new series of single-use plastic anastomats, which have been widely used clinically. The GF-1 type tubular anastomat, XF stump anastomat, CF side-to-side anastomat, and anastomosis forceps are manufactured in China. Gastrointestinal anastomats are all designed based on staplers. The mechanical parts of the anastomats are equipped with “Π”-shaped staples similar to staplers. The thrust acting on the “Π”-shaped staples pushes them through the gastrointestinal wall tissue. Next, the staples are bent into a “B” shape, stapling the gastrointestinal tissue together. Anastomats manufactured in China use tantalum wires (0.2˜0.3 mm diameter) as the suture material. This material is compatible with human tissue and can be left in the human body long-term with no adverse reactions.

16—A pressure sensor chip using semiconductor or piezoelectric elements—sensors that convert pressure to an electrical signal (a readily available pressure sensor may also be used, but the strain force needs to be removed and attached to the side of the stapling apparatus and staple holder). The strain, which corresponds to the pressure of the stapling apparatus and staple holder, is transmitted out by a regular bridge circuit. The trigger will eject staples after the adjusting screw is fixed according to the pressure of the empennage screw knob and adjusting screws. It will mount on either side or both sides of the anastomat stapling apparatus and staple holder. It will also adjust, amplify, and display the signal from the pressure sensor.

The amplifying circuit includes an operational amplifier and displays the pressure value on the operation room display screen through wireless communication after the AD conversion circuit and digital display circuit.

Like the cylindrical anastomat, the pressure values are transmitted to a monitor on the handle through the center rod. The operator can directly understand the pressure at anastomosis and determine whether the existing pressure is appropriate. Furthermore, the stapling apparatus and staple holder of the anastomat close progressively from the center to the periphery and control the proximal and distal pressure from the pressure sensor.

1. An anastomat and closure device having a pressure sensor and automatic adjustment means, characterized in that the pressure value from direct or indirect contact of a stapling apparatus and a staple holder can be transmitted and displayed by a pressure sensor.

2. The anastomat and closure device described in claim 1, characterized in that the pressure sensor is installed on either side or on both sides of the stapling apparatus and staple holder and wherein the pressure is displayed through a pressure sensor shaping circuit, an amplifying circuit, and a display circuit.

3. The anastomat and closure device described in claim 2, wherein the amplifying circuit uses an operational amplifier, and the value is displayed after the operation of AD conversion circuit and the digital display circuit and signals are transmitted wirelessly and are displayed on a screen in the operation room.
